(19) United States
(12) Patent Application Publication
(10) Pub. No.: US 2008/0269544 A1
(43) Pub. Date: Oct. 30, 2008

(54) Snooze Ezz

(76) Inventors: Mark R. Atkin, Hull (GB); Robert Atkin, Hull (GB)

Correspondence Address: Raymond M. Galasso;Galasso & Associates LP, P.O. Box 26503, Austin, TX, 78755-0503, US

(21) Appl. No.: 11/796,470

(22) Filed: Apr. 27, 2007

(51) Int. Cl. A61M 21/00

(52) U.S. Cl. 600/26

(57) ABSTRACT

Snooze Ezz is a device to aid an individual in sleeping while in a seated position. One embodiment of the invention utilizes a chin cup, adjustable straps with hangers, quick release clips and ear pads. Other embodiments of the invention may include earplugs on the adjustable straps. Still other embodiments of the invention may utilize velcro hook and loop arrangements instead of hangers. To use the preferred embodiment of Snooze Ezz, a user would simply attach the hangers to a seatback or other suitable location. He would then slip his head between the adjustable straps and position the chin cup under his chin. The individual can also slip the ear pads and/or earplugs on to block out light or noise.

### CROSS REFERENCE TO RELATED APPLICATIONS

This United States Non-Provisional Patent Application does not claim priority to any U.S. Provisional Patent Application or any foreign patent application.

### FIELD OF THE DISCLOSURE

The disclosures made herein relate generally to the travel accessories industry. The invention discussed herein is in the general classification of travel sleeping devices.

### BACKGROUND

Medical studies have proven that the amount of sleep an individual receives each night is related to the person's overall health. Medical professionals recommend the average person sleep for eight or nine hours each night in order to provide the body with enough rest to function properly. A sufficient amount of sleep also improves a person's ability to think clearly and quickly, thus allowing the individual to be more effective with the tasks undertaken throughout the course of the day.

People, however, are not always fortunate enough to be able to sleep in their beds when sleep is necessary. Individuals that travel for business or simply travel for pleasure on buses, planes or in a car often find themselves attempting to get sleep at awkward times. Most of this travel sleep is done in a sitting position in an airplane seat or in the passenger seat of an automobile or bus. While sleep can be attained in these positions, it is not always comfortable and can result in stiff and painful muscles due to unnatural positioning of a person's head, neck and back.

Small pillows designed to help cushion a person's head and neck are often employed to mitigate the discomfort associated with sleeping in a seated position. However, small pillows are not always available or effective. An individual still may experience a level of discomfort preventing suitable rest even when small pillows are used.

Other devices such as horseshoe shaped pillows and sling type contraptions also have been invented as aides for seated sleeping. Horseshoe shaped pillows often shift during travel, preventing an individual from maintaining a comfortable position for an extended period. The sling type contraptions are also unwieldy and aesthetically unappealing.

Hence, there is a need in the art for a convenient to use, inexpensive, durable, safe, aesthetically appealing and effective device for giving an individual sufficient support to sleep comfortably in a seated position.

### SUMMARY OF THE DISCLOSURE

Snooze Ezz is a device to aid an individual in sleeping while in a seated position.

One embodiment of the invention utilizes a chin cup, adjustable straps with hangers, quick release clips and ear pads. Other embodiments of the invention may include earplugs or eye pads attached to the adjustable straps. Still other embodiments of the invention may utilize a velcro hook and loop arrangement instead of a hanger.

The principal object of this invention is to provide a device that is capable of supporting an individual's neck and head when he is in a seated position to minimize shoulder and neck stiffness while sleeping.

Another object of this invention is to provide a device that is aesthetically appealing and capable of supporting an individual's neck and head when he is in a seated position to minimize shoulder and neck stiffness while sleeping.

Another object of this invention is to provide a portable device for supporting an individual's neck and head when he is in a seated position to minimize shoulder and neck stiffness while sleeping.

Another object of this invention is to provide an affordable device for supporting an individual's neck and head when he is in a seated position to minimize shoulder and neck stiffness while sleeping.

Another object of this invention is to provide a safe device for supporting an individual's neck and head when he is in a seated position to minimize shoulder and neck stiffness while sleeping.

Yet another object of this invention is to provide a durable device for supporting an individual's neck and head when he is in a seated position to minimize shoulder and neck stiffness while sleeping.

### BRIEF DESCRIPTION OF THE DRAWINGS

FIG. 1 depicts a perspective view of the preferred embodiment of the present invention.

FIG. 2 depicts a close-up view of an alternative embodiment of the adjustable straps of the present invention.

### DETAILED DESCRIPTION OF THE DRAWINGS

The preferred embodiment of Snooze Ezz is comprised of at least some of the following: a chin cup, adjustable straps with quick release clips, hangers and ear pads.

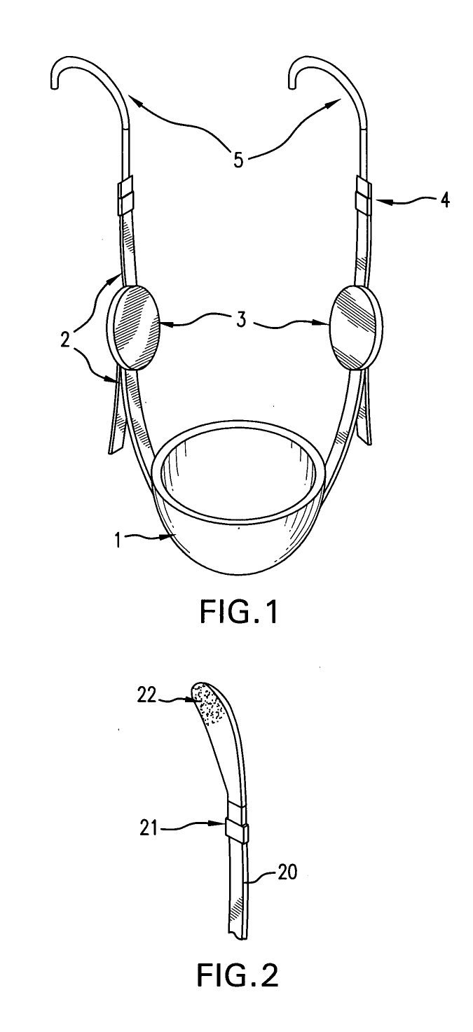

FIG. 1 shows the preferred embodiment of the present invention. A chin cup 1 that is shaped like a bowl is attached to adjustable straps 2. The chin cup 1 is made of plastic in this preferred embodiment though a variety of materials may be utilized. The chin cup 1 has a depth of approximately two inches and a width of approximately six inches in this preferred embodiment.

Ear pads 3 are attached at approximately the midpoint of each one of the adjustable straps 2. The ear pads 3 are circular with a diameter of approximately four inches in this preferred embodiment. The ear pads 3 are covered with a soft cloth material and are made of a dark color in the preferred embodiment.

A quick release clip 4 is located above the ear pads 3 on the adjustable straps 2 to allow the adjustable straps 2 to be extended or shortened to accommodate different users. A hanger 5 is attached to the top of each one of the adjustable straps 2. The hanger 5 is made of plastic and has a semicircular shape. The quick release clip 4 is made of plastic in this preferred embodiment though metal or other materials may also be utilized.

The adjustable straps 2 are made of nylon and are capable of extending between six inches and eighteen inches in length in this preferred embodiment. The adjustable straps 2 are approximately one half of an inch wide and one-eighth of an inch in depth. Rubber or other materials may also be used for the adjustable straps.

FIG. 2 shows a close-up view of an alternative embodiment of the adjustable straps of the present invention. The adjustable strap 20 has the same type of quick release clip 21 on it as the ones shown in FIG. 1. However, the adjustable strap 20 has a velcro hook and loop fastener 22 on the top to allow it to attach to the back of a fabric seat or other similar materials instead of the hanger used as a fastening mechanism in FIG. 1.

Other embodiments of the invention also have earplugs attached to the adjustable straps to allow a user to prevent noise from entering the user's ears. Ear pieces that permit music to be listened to could also accompany the device. Eye pads could also be attached to the adjustable straps in some embodiments.

To use the preferred embodiment of Snooze Ezz, a user would simply attach the hangers to a seatback or in another desired location. He would then slip his head between the adjustable straps and position the chin cup under his chin. The adjustable straps can be adjusted to fit all sized users. The individual can also slip the ear pads and/or earplugs on to block out noise. When the user is finished with the device, it easily can be placed into a carry-on bag or purse.

The materials utilized for Snooze Ezz may vary widely but will likely include metals and or plastic. The metals would ideally be selected from available steel or alloys of steel and aluminum. The production process related to the use of these metals insures that the metal is non-corrosive, durable and strong. The selected metal should have high impact strength and be capable of accepting and retaining coloring materials for an extended length of time.

The plastic used in the production will ideally be selected for durability and longevity. Thermoplastics are commonly used in the manufacturing of components similar to those used in this invention. Polyethylene, polypropylene, and other similar thermoplastic materials would be among those with the necessary traits. Members of this family are recognized universally as being versatile and of high quality.

The plastic components of Snooze Ezz can also be formed with the use of plastic molding techniques, such as injection molding or blow molding. Injection molding requires melted plastic to be forcefully injected into relatively cool molds. As the plastic begins to harden, it takes on the shape of the mold cavity. This technique is ideal for the mass production of products. Alternatively, blow molding, a form of extrusion, could be utilized. Blow molding involves a molten tube being pushed into a mold. Compressed air then forces the molten tube against the cold walls of the mold.

It should be obvious that the components of the present invention can be of various shapes and sizes. It should also be obvious that the components of the invention can be made of different types of plastics or other suitable materials and can be of any color.

It will be recognized by those skilled in the art that changes or modifications may be made to the above-described embodiments without departing from the broad inventive concepts of the invention. It should therefore be understood that this invention is not limited to the particular embodiments described herein, but is intended to include all changes and modifications that are within the scope and spirit of the invention as set forth in the claims.

What is claimed is:

1. A sleep supporting device comprising:
(a) a chin cup attached to a first strap and a second strap; and
(b) a fastening mechanism attached to the top of the first strap and the second strap opposite the chin cup.

2. The device of claim 1 wherein the chin cup is shaped like a bowl.

3. The device of claim 1 wherein the chin cup is made of plastic.

4. The device of claim 1 wherein the chin cup has a depth of approximately two inches and a width of approximately six inches.

5. The device of claim 1 wherein the first strap and the second strap are adjustable.

6. The device of claim 1 wherein the first strap and the second strap are approximately one half of an inch in width and one-eighth of an inch in depth and eighteen inches in length.

7. The device of claim 1 wherein the first strap and the second strap are made of nylon.

8. The device of claim 1 further comprising
a first ear pad attached to the first strap and a second ear pad attached to the second strap.

9. The device of claim 8 wherein the first ear pad and the second ear pad are circular.

10. The device of claim 9 wherein the first ear pad and the second ear pad have a diameter of approximately four inches.

11. The device of claim 8 wherein the first ear pad and the second ear pad are covered with a soft dark material.

12. The device of claim 1 wherein a first quick release clip is located on the first strap and a second quick release clip is located on the second strap.

13. The device of claim 1 wherein the fastening mechanism is a first hanger at the top of the first strap and a second hanger at the top of the second strap.

14. The device of claim 13 wherein the first hanger and the second hanger are made of plastic.

15. The device of claim 1 wherein the fastening mechanism is a first velcro hook and loop fastener on the top of the first strap and a second velcro hook and loop fastener on the top of the second strap.

16. The device of claim 1 further comprising
a first earplug attached to the first strap and a second earplug attached to the second strap.

17. A sleep supporting device comprising:
(a) a chin cup made of plastic attached to a first adjustable strap made of nylon and a second adjustable strap made of nylon;
(b) a first hanger made of plastic attached to the top of the first adjustable strap and a second hanger made of plastic attached to the top of the second adjustable strap;
(c) a first ear pad that is approximately circular and made of a soft and dark material attached to the first adjustable strap and a second ear pad that is approximately circular and made of a soft and dark material attached to the second adjustable strap;
(d) a first quick release clip located on the first adjustable strap and a second quick release clip located on the second strap; and
(e) a first earplug attached to the first adjustable strap and a second earplug attached to the second adjustable strap.
